(19) United States
(12) Patent Application Publication
(10) Pub. No.: US 2018/0344749 A1
(43) Pub. Date: Dec. 6, 2018

(54) CONTROLLED RELEASE FORMULATIONS OF SALICLATE-RELEASING ACTIVES

(71) Applicant: Cogwellin, LLC, Eatontown, NJ (US)

(72) Inventors: Joel S. Ross, Monmouth Beach, NJ (US); Bruce N. Rehlaender, Lake Oswego, OR (US)

(21) Appl. No.: 15/771,704

(22) PCT Filed: Dec. 9, 2016
(86) PCT No.: PCT/US16/65809

§ 371 Date: Apr. 27, 2018

Related U.S. Application Data
(60) Provisional application No. 62/265,634, filed on Dec. 10, 2015.

(51) Int. Cl. A61K 31/621; A61K 9/16; A61K 9/24; A61K 9/28; A61P 25/28

(52) U.S. Cl. CPC A61K 31/621; A61K 9/167; A61P 25/28; A61K 9/2833; A61K 9/209

(57) ABSTRACT

This invention relates to pharmaceutical controlled release compositions of salicylate-releasing actives for the treatment of tau protein-related neurodegenerative diseases and disorders.

### BACKGROUND

Alzheimer's disease is the sixth-leading cause of death in the United States. More than 15 million Americans provide unpaid care for individuals with Alzheimer's or another dementia, and payments for the health care of dementia patients are estimated to be $226 billion in 2015. If the onset of Alzheimer's disease could be delayed by only five years, the number of individuals with Alzheimer's disease could be reduced by as much as 50% over time.

Previously it has been shown that anti-inflammatory medications have an inverse association with Alzheimer's, improve cognitive performance, and slow decline in patients with Alzheimer's disease. (Broe, et al. Anti-inflammatory Drugs Protect Against Alzheimer's Disease at Low Doses. Arch Neurol. 57, 1568-1591 (2000)).

The accumulation of Tau fibrils in the brain characterizes a group of neurodegenerative diseases known as tauopathies, which include frontotemporal dementia (FTD) and Alzheimer's disease. Although the mechanism by which soluble Tau protein accumulates to form insoluble Tau fibrils is not fully understood, Tau protein acetylation has been associated with the progression of tauopathies.

U.S. Publication No. 2013/0251731 describes methods of diagnosing tauopathies using Tau protein acetylation as a biomarker for these diseases. In particular, acetylation of the Tau protein on at least one of the following lysine residues is suggested as biomarker for certain tauopathies: K150, K163, K174, K234, K240, K259, K274, K280, K281, K290, K311. K369, and K395. Furthermore, studies of the brains from Alzheimer's disease patients and related tauopathies show that acetylated K280 Tau is specifically associated with insoluble Tau aggregates. In these studies, Tau K280 acetylation was only detected in diseased tissues, suggesting that acetylated K280 Tau may have a role in causing insoluble Tau accumulation. As such, acetylated K280 Tau is considered a specific biomarker for Alzheimer's disease and other neurodegenerative disorders.

Furthermore, other studies suggest that acetylation of soluble tau species could occur at an early stage in the disease, and that inhibition of this process could be a potential therapeutic strategy. It has been shown that acetyltransferase p300-induced tau acetylation can be inhibited by salsalate and salicylate, which enhance tau turnover and reduce tau levels. In the PS19 transgenic mouse model of FTD, administration of salsalate after disease onset inhibited p300 activity, lowered levels of total tan and tau acetylated at K174, rescued tau-induced memory deficits and prevented hippocampal atrophy. In cell cultures, unlike salsalate, aspirin was found to rapidly lose its acetyl group and boost tau acetylation. It has been also shown that salsalate penetrates the brain and gives rise to relatively stable salicylate levels over 8 hours. (Min, et al. Critical role of acetylation in tau-mediated neurodegeneration and cognitive deficits. Nature Medicine, 21, 1154-1162 (2015)).

Accordingly, salicylate inhibition of tau acetylation could be a new therapeutic strategy against human tauopathies. The oral salicylate dosage forms of the present disclosure are effective in treating tauopathies such as FTD and Alzheimer's disease, and provide a continuing effect for up to 24 hours post-dosing.

### SUMMARY OF THE INVENTION

The present disclosure, in various embodiments, is directed to an oral pharmaceutical composition comprising at least one salicylate-releasing immediate release component; and at least one salicylate-releasing delayed release component; wherein after ingestion the oral pharmaceutical composition maintains a plasma concentration of salicylate ranging from about 1 to about 3 μg/mL over a period of at least about 12 hours.

In some of the embodiments of the present disclosure, the oral pharmaceutical composition comprises at least two different delayed release components.

Some embodiments of the present disclosure relate to the oral pharmaceutical composition comprising a single immediate release component.

In certain embodiments, the oral pharmaceutical composition comprises a single immediate and two different delayed release components.

The oral pharmaceutical composition can be in the form of particles, wherein the two different delayed release components each comprise a plurality of particles comprising a salicylate-releasing active ingredient coated with a coating comprising a pharmaceutically acceptable enteric polymer, and the immediate release component comprises a plurality of particles comprising a salicylate-releasing active ingredient free of any coating which substantially delays release of salicylate.

In various embodiments the oral pharmaceutical composition can be in the form of particles selected from the group consisting of beads, granulates, pellets, minitabs, or mini tablets.

In some embodiments, the oral pharmaceutical composition of the present invention comprises at least one salicylate-releasing immediate release component and at least one salicylate-releasing delayed release component, each comprising a salicylate-releasing active ingredient selected from the group consisting of salsalate, salicylic acid, and salts or esters thereof.

In one of the embodiments, the salicylate-releasing active ingredient is salsalate.

Some embodiments of the present disclosure relate to the oral pharmaceutical composition wherein the two different delayed release components are each coated with a coating comprising a pharmaceutically acceptable enteric polymer.

In some embodiments, the pharmaceutically acceptable enteric polymer of at least one delayed release component substantially dissolves at about pH 6. In some embodiments, the pharmaceutically acceptable enteric polymer of at least one delayed release component substantially dissolves at about pH 7.

In certain embodiments where the oral pharmaceutical composition comprises two different delayed release components, the pharmaceutically acceptable enteric polymer of one delayed release component substantially dissolves at about pH 6, and the pharmaceutically acceptable enteric polymer of the other delayed release component substantially dissolves at about pH 7.

One of the embodiments of the present disclosure relates to an oral pharmaceutical composition wherein the amount of salsalate in the immediate release component is about 20 to about 40 wt. %, the amount of salsalate in the delayed release component coated with pharmaceutically acceptable enteric polymer that substantially dissolves at about pH 6 is about 10 to about 30 wt. %, and the amount of salsalate in the delayed release component coated with pharmaceutically acceptable enteric polymer that substantially dissolves at about pH 7 is about 40 to about 60 wt. %, wherein the respective wt. % are based on the total amount of salsalate in the oral pharmaceutical composition.

In one of the embodiments, the oral pharmaceutical composition comprises a total dose of salsalate ranging from about 200 to about 400 mg.

Some embodiments of the present disclosure relate to a method of inhibiting tau protein acetylation, e.g. in the brain of a patient, comprising orally administering the oral pharmaceutical composition of the present invention to a patient in need thereof.

Some embodiments relate to a method of treating a neurodegenerative disease or disorder associated with the pathological aggregation of tau protein in the brain comprising orally administering the oral pharmaceutical composition of the present invention to a patient in need thereof.

The present disclosure relates to compositions and treatments for neurodegenerative diseases or disorders selected from the group consisting of Alzheimer's disease, Amyotrophic lateral sclerosis-Parkinsonism-dementia (ALS/PD) complex of Guam, Argyrophilic grain disease, British type amyloid angiopathy, Corticobasal degeneration, Dementia pugilistica (TBI), Down's syndrome, Frontotemporal dementia, Gerstmann-Straussler-Scheinker Syndrome, Hallenvorden-Spatz disease. Inclusion body myositis, Multisystem atrophy, Myotonic dystrophy, Niemann-Pick disease type C, Parkinson with dementia of Guadeloupe, Pick's disease, Presenile dementia with tangles, Prion protein amyloid angiopathy, Progressive supranuclear palsy, Post-encephalitic parkinsonism, Subacute sclerosing panencephalitis, and Tangle only dementia.

In some embodiments, the neurodegenerative disease or disorder is Alzheimer's disease.

Unless otherwise defined, all technical and scientific terms used herein have the same meaning as commonly understood by one of ordinary skill in the art to which this disclosure pertains. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference herein, for all purposes. The references cited herein are not admitted to be prior art to the claimed invention. In addition, the materials, methods, and examples are illustrative only and are not intended to be limiting.

Other features and advantages of the present disclosure will be apparent from the following detailed description and claims.

### BRIEF DESCRIPTION OF THE DRAWINGS

FIG. 1A shows the results of Western blot analysis performed to detect acetylated 280 Tau protein in the cerebrospinal fluid of a control patient (Lane 2) and a patient with Alzheimer's disease (Lane 3).

FIG. 1B shows the results of Western blot analysis performed to detect acetylated 280 Tau protein in the cerebrospinal fluid of a control patient (Lane 2) and a patient with Alzheimer's disease (Lane 3) using a lighter stain than was used in the Western blot analysis of FIG. 1A.

FIG. 2A shows the results of immunohistochemical staining performed to detect acetylated 280 Tau protein in the white matter at the superior cerebellar peduncle of a control patient.

FIG. 2B shows the results of immunohistochemical staining performed to detect acetylated 280 Tau protein in the white matter at the superior cerebellar peduncle of a Braaks Stage 1 AD patient.

FIG. 3A shows the results of immunohistochemical staining performed to detect acetylated 280 Tau protein in the cerebral white matter of a control patient.

FIG. 3B shows the results of immunohistochemical staining performed to detect acetylated 280 Tau protein in the cerebral white matter of a Braaks Stage 1 AD patient.

FIG. 4A shows the results of immunohistochemical staining performed to detect acetylated 280 Tau protein in the putamen of a Braaks Stage 1 AD patient.

FIG. 4B shows the results of immunohistochemical staining performed to detect acetylated 280 Tau protein in the neurons of the putamen of a Braaks Stage 1 AD patient.

FIG. 5A shows the results of immunohistochemical staining performed to detect acetylated 280 Tau protein in the caudate nucleus of a Braaks Stage 1 AD patient.

FIG. 5B shows the results of immunohistochemical staining performed to detect acetylated 280 Tau protein in the large cholinergic interneurons in the caudate nucleus of a Braaks Stage 1 AD patient.

FIG. 6A shows the results of immunohistochemical staining performed to detect acetylated 280 Tau protein in the reticular thalamic nucleus of a Braaks Stage 1 AD patient.

FIG. 6B shows the results of immunohistochemical staining performed to detect acetylated 280 Tau protein in the neurons of the reticular thalamic nucleus of a Braaks Stage 1 AD patient.

FIG. 7 provides a PK model of the sum of salsalate and salicylate plasma concentrations over time after administration of a single dose of an immediate release salsalate dosage form.

FIG. 8A shows the target salicylate plasma profile of an exemplary “ideal” composition which can provide a target PK profile of about 2 μg/mL plasma levels of salicylate over a 24 hour period.

FIG. 8B shows the target “ideal” release profile, expressed as the relative percentage of drug released as a function of time after administration, which provides about 2 μg/mL plasma levels of salicylate over a 24 hour period. The drug is released in multiple pulses.

FIG. 9A provides a comparison of the targeted “ideal” (dashed line) and a “realistic” (solid line) PK profile achievable by the salicylate-releasing composition according to the present disclosure.

FIG. 9B provides a comparison of the target “ideal” (dashed line) and realistic (solid line) release profiles of a salicylate-releasing composition according to the present disclosure.

FIG. 10 provides drug release profile of an exemplary dosage form containing 200 mg of salsalate.

### DETAILED DESCRIPTION OF THE INVENTION

The present disclosure is directed, in various embodiments, to oral pharmaceutical compositions comprising at least one salicylate-releasing immediate release component: and at least one salicylate-releasing delayed release component; wherein after ingestion the oral pharmaceutical composition maintains a plasma concentration of salicylate ranging from about 1 to about 3 μg/mL over a period of at least about 12 hours.

As used herein, the “drug” or “salicylate drug” or “salicylate” or salicylic compound” refers to a salicylate-releasing active(s) or salicylic compound(s) which includes pharmaceutically acceptable salts, esters, ethers, and other derivatives of salicylic acid which after ingestion metabolize to salicylic acid.

Salicylic compounds are readily absorbed from the stomach. Gastric absorption is more rapid at lower pH. The rate of intestinal absorption of salicylic compounds is less affected by the pH of the solution than is their rate of gastric absorption. Gastric and intestinal absorption are more rapid with the smaller salicylic molecules (e.g., sodium salicylate, salicylic acid, and calcium salicylate) than acetylsalicylic acid. (Bradley et al. The Rate of Absorption of Salicylates and the Effect of Certain Compounds on the Rate of Absorption of Acetylsalicylic Acid From the Stomach and Intestine. Am J Dig Dis. 3(6), 415-419 (1936))

Salicylates such as salsalate (salicylsalicylic acid) are Nonsteroidal Anti-Inflammatory Drugs (NSAIDs). Salsalate, a dimer of salicylic acid, has been widely used as an analgesic and anti-inflammatory agent for about 100 years. The drug is well tolerated and considered safe after decades of clinical use.

Salsalate is a derivative of salicylic acid which on hydrolysis yields two molecules of salicylic acid. The slightly lower levels of salicylic acid (in terms of salicylic acid equivalents) in plasma after salsalate administration compared to that found after aspirin administration may reflect direct biotransformation of some of the salsalate to salsalate conjugates (e.g., glucuronide conjugates), without hydrolysis to salicylic acid.

Based on relative AUC values for salicylic acid, salsalate is hydrolyzed in the body to give approximately 84% of the salicylic acid obtained from an equivalent dose (in terms of salicylic acid equivalents) of aspirin (acetylsalicylic acid). It has been also found that salsalate causes significantly less gastrointestinal bleeding and gastric erosions at anti-inflammatory doses than does aspirin (Harrison et al. Absorption, Biotransformation, and Pharmacokinetics of Salicylsalicylic Acid in Humans. J Clin Pharmacol. 21, 401-404 (1981)). The reduction in adverse gastric effects has been attributed to the differences in solubility for the two agents; salsalate undergoes particulate dispersion in the pH of the stomach and is soluble in the pH of the small intestine, while aspirin is soluble in the acidic pH of the stomach (Harrison et al. Effect of Food on Salsalate Absorption. Therapeutic Drug Monitoring. 14, 87-91 (1992)).

“Immediate-release” (IR) or “rapid release” (RR), which are used interchangeably herein, refers to dosage forms or drug-containing formulations, or drug-containing components of such formulations (e.g., an immediate release population of drug particles, an immediate release drug layer, etc.) in which the rate of release of drug from the formulation (e.g., particles) is neither appreciably nor intentionally retarded by manipulating the composition or structure of the formulation. For example, an immediate release composition or particle would not include a sustained, delayed, or extended release coating or drug matrix intended to appreciably delay or retard release or dissolution of the drug from the composition. Without limitation, an immediate release formulation or particle may, for example, release greater than or equal to about 50%, in some embodiments greater than about 75%, greater than about 80%, or more than about 90%, and in certain embodiments greater than about 95% of the drug within about 30 minutes when dissolution tested using United States Pharmacopoeia (USP <711>) dissolution methodology (Apparatus 2-paddles@ 50 RPM, 0.1N HCl at 37° C.).

IR/RR components or particles can include, but are not limited to particles of the drug itself, formulations in which the drug is layered onto an inert core such as sugar spheres, cellulose spheres, cellulose-lactose spheres, etc., granulates of the drug with powdered excipients such as pharmaceutically acceptable fillers, diluents, glidants, etc., extruded and optionally spheronized combinations of drug and one or more excipients, and so forth. The inert core (e.g., a sugar sphere) layered with a drug may further comprise an optional binder. In some cases, the immediate release components can be coated with an optional non-functional, protective sealant coating.

Non-limiting examples binders or polymeric binders including film-forming binders that are used to bind the drug to the inert sugar sphere are usually water-soluble, alcohol-soluble or acetone/water soluble binders, such as polyvinylpyrrolidone (PVP), polyethylene oxide, hydroxypropyl methylcellulose (HPMC), or hydroxypropylcellulose (HPC), and may be used at concentrations of about 0.5 to about 10 weight % based on the drug-layered beads. The drug substance may be present in this coating formulation in solution form or may be suspended at a solid content up to about 35% by weight depending on the viscosity of the coating formulation. Examples of useful commercially available polymeric binders include, but are not limited to, hydroxypropylcellulose (e.g., Klucel® LF from Aqualon), starches including modified starch (e.g., Starch 1551 and Starch 1500, commercially available from National Starch and Colorcon, respectively), Kollidon®) VA 64, poly(vinylacetate-vinyl pyrrolidone) from BASF, and hydroxypropyl methylcellulose (for example, with a viscosity of 100 cps or more, e.g., Methocel K100LV and Metolose K400 commercially available from Dow Chemical and Shin Etsu Chemicals, respectively) alone or in combination with a widely used binder such as PVP (polyvinylpyrrolidone) or hydroxypropyl methylcellulose (for example, with a viscosity of 15 cps or less).

Drug microgranules containing milled or micronized drug may be produced by granulating the active and a suitable filler/diluent (if required) with a polymeric binder in a high-shear granulator.

Polymeric binders may also impart resilient characteristics to the dried microgranules to resist attrition due to stirring during further processing including, for example, solvent coacervation for taste-masking.

The relative amounts of active and optional filler/diluent may vary considerably depending on the particular active and the dosage form. In some embodiments, drug-containing microgranules can contain from about 20% to about 99% active, and up to about 15% binder with any optional filler/diluent being present at from about 0 to 80% by weight of granules (microgranules).

IR/RR components or particles can be uncoated, or coated with a non-functional coating. Non-functional coatings are film coatings applied for reasons of improved appearance, improved handling, and prevention of dusting, protection from contact with other components of the formulation, etc. Non-functional coatings are distinguished from functional film coatings which are applied to modify the release of the drug (e.g., delay and/or extend release). Examples of polymers useful in non-functional coatings include but are not limited to cellulose ethers such as hydroxypropylmethyl cellulose (HPMC), methyl cellulose (MC), hydroxyethyl cellulose (HEC), hydroxypropyl cellulose (HPC), etc. While such polymers may also be useful in functional coatings, in nonfunctional coatings the polymers are suitably plasticized or applied as a thin layer so as to minimize the effect on drug release.

In one embodiment, the non-functional coating is a sealant coating comprising a hydrophilic polymer. Non-limiting examples of suitable commercially available hydrophilic polymers include hydrophilic hydroxypropylcellulose (e.g., Klucel® LF), hydroxypropyl methylcellulose or hypromellose (e.g., Opadry® Clear or Pharmacoat™ 603), vinylpyrrolidone-vinylacetate copolymer (e.g., Kollidon® VA 64 from BASF), and low-viscosity ethylcellulose (e.g., viscosity of 10 cps or less a 5% solution in 80/20 toluene/alcohol at 25° C. as measured using an Ubbelohde viscometer).

The non-functional or sealant layer can constitute from about 1% to about 20% of the weight of the drug-containing, sealant-coated core, for example about 1%, about 2%, about 3%, about 4%, about 5%, about 7%, about 10%, about 12%, about 15%, about 17%, or about 20%, inclusive of all ranges and subranges therebetween.

The term, “delayed release” (DR), as defined herein, refers to a type of release in which a pulse of the drug (e.g., IR drug particles or component) is released after a pre-determined lag time. The term “lag-time” refers to a time period during which little or no drug is released from the drug-containing components, for less than about 10%, less than about 5%, or more particularly substantially none, of the drug is released. Such a lag-time can be from as little as 1 to 2 hours after administration or as long as 10-15 hours after administration, including lag times of about 1, about 2, about 3, about 4, about 5, about 6, about 7, about 8, about 9, about 10, about 11, about 12, about 13, about 14, or about 15 hours, inclusive of all ranges and subranges therebetween.

The lag time between the pulses can be modified depending on the desired pharmacokinetic profile and dosing regimen. The lag time can be modified based on the choice of the coating, coating weight, and any additives included in the coating which can either promote or impede the dissolution of the drug at the desired pH of the gastrointestinal tract.

Various approaches can be taken to providing and controlling such lag times, including, for example coating the drug-containing particles with enteric polymers, or combinations of enteric polymers with other types of polymers such as water-soluble, water-insoluble, and gastrosoluble polymers. The lag time can be further controlled by, for example, the specific choice of polymer(s), coating thickness, addition of plasticizers or pore formers, etc.

Non-limiting examples of suitable gastrosoluble pore-formers include, e.g. calcium carbonate, calcium phosphate, calcium saccharide, calcium succinate, calcium tartrate, ferric acetate, ferric hydroxide, ferric phosphate, magnesium carbonate, magnesium citrate, magnesium phosphate, magnesium hydroxide and mixtures thereof. Non-limiting examples of suitable water-soluble pore-formers include, e.g. sodium chloride, sucrose, and povidone.

A delayed release coating or delayed release layer refers to a layer, membrane, or coating which provides such properties. A delayed release component or particle(s) can be an IR component or particle(s) further coated with an enteric coating. As is understood in the art, enteric coatings typically comprise polymers having pH-dependent solubility such that the polymer is less soluble at low pH, and more soluble at high pH.

The in vitro drug release profiles of delayed release (or sustained release or a combination of sustain and delayed release) particles may be determined by dissolution testing in a USP <711> Apparatus 1 or 2, particularly Apparatus 2 (baskets), using a two-stage or three-stage dissolution medium (first 2 hours in 700 mL of 0.1N HCl at 37° C. followed by dissolution testing at pH 6 for 2 hours, for example obtained by the addition of 200 mL of a pH modifier, and then by dissolution testing at pH 7.4 obtained by the addition of another 200 mL of a pH modifier). Drug release with time can be determined using various methods, for example by HPLC on samples pulled at selected time points.

According to USP <711>, a two-step dissolution method is performed to determine the integrity of the enteric coating in an acidic environment and to measure the release of the dosage form in a neutral environment. For immediate-release solid, oral dosage forms that are enteric-coated, USP requires a two-step dissolution method that demonstrates coating integrity in an acidic environment for two hours, followed by exposure to a neutral pH environment for about one hour. The test begins with the dosage form in an acidic environment (USP requires 0.1N HCl), followed by a pH shift to a neutral environment. The release can be measured in a medium with a buffer, e.g., phosphate buffer, at pH 6-7.4. In addition to delayed-release formulations, a two-step dissolution method is also needed to determine the integrity of enteric-coated, extended-release solid oral dosage formulations. In this case, the coating integrity is demonstrated in an acidic environment for two hours followed by release in a neutral pH environment, typically for up to 24 h. Since enteric-coated, extended-release solid oral dosage formulations target the lower GI tract, it is important that the release be observed during the buffer stage.

A non-limiting list of enteric polymers suitable for use in the DR layer includes cellulose acetate phthalate, hydroxypropyl methylcellulose (hypromellose) phthalate, hydroxypropyl methylcellulose (hypromellose) acetate succinate, polyvinyl acetate phthalate, pH-sensitive methacrylic acid/methylmethacrylate copolymers (e.g., EUDRAGIT® L, S and FS polymers), shellac, etc. and mixtures thereof. Some commercially available materials that may be used are, for example, methacrylic acid copolymers sold under the trademark Eudragit (L100, S 100, L30D) from Evonik, Cellacefate (cellulose acetate phthalate) from Eastman Chemical Co., Aquateric (cellulose acetate phthalate aqueous dispersion) from FMC Corp., and Aqoat (hydroxypropyl methylcellulose acetate succinate aqueous dispersion) from Shin Etsu K.K.

Anionic enteric polymers with carboxyl groups have higher water solubility at basic pH than at acidic pH. These polymers can be used for preventing gastric degradation of drug, colon drug delivery and achieving high bioavailability of weakly basic drugs. Poly(methacrylic acid-co-methyl methacrylate) (Eudragit L, S and F), hydroxypropylmethylcellulose phthalate (HPMC-P) and HPMC acetate succinate (HPMC-AS), which possess carboxyl groups on the polymer side chains, are insoluble at stomach low pH but soluble at intestinal neutral pH. The pH value controlling the aqueous solubility of the polymers can be finely tuned by adjusting the amount of carboxyl or other substituent groups on the polymers. The ratio of carboxyl groups to ester groups (carboxyl/ester ratio) of poly(methacrylic acid-co-methyl methacrylate) can be manipulated to control the polymer dissolving pH.

EUDRAGIT® L and S polymers enable targeting specific areas of the intestine. There is a broad product portfolio of anionic EUDRAGIT® grades which dissolve at rising PH values. In addition, the different grades can be combined with each other, making it possible to adjust the dissolution pH, and thus to achieve the required GI targeting for the drug.

Colon drug delivery can also be achieved using enteric polymers. The outermost enteric polymer coat prevents the dissolution of the acid-soluble polymer in the acidic gastric environment but dissolves in the more basic intestinal environment.

For example, Eudragit L has a carboxyl/ester ratio of 1:1 and is soluble at pH 6, while Eudragit S having a ratio of 1:2 is soluble at pH 7. By varying the amount of phthalate groups, HPMC-P can be designed to dissolve at pH 5.0-5.5. A high polymer dissolving pH (pH 5.5-6.8) can be achieved by HPMCAS through adjusting the amounts of methoxyl, hydroxypropyl, acetyl and succinoyl groups on the polymers. These polymers are the FDA-approved enteric polymers and are widely used for pharmaceutical products on the market.

Eudragit F is another example of enteric polymers, which due to its solubility at pH higher than 7.0 allows for colon-targeted drug delivery. Eudragit F and also chitosan are designed to dissolve in the small intestine and the colon, respectively.

The term “sustained-release coating” or “SR coating” refers to a coating providing sustained release properties, e.g. a coating which slows the release of the drug from the drug-containing particle but does not provide an appreciable “lag-time.”

In one embodiment, the SR coating comprises a water-insoluble polymer and optionally a water-soluble polymer.

Water-soluble and water-insoluble polymers have pH-independent solubility, whereby water-soluble polymers have relatively high solubility over a wide range of pHs, and conversely water-insoluble polymers have a relatively low solubility over a wide range of pHs. Gastrosoluble polymers have pH-dependent solubility such that they are more soluble at low pH than high pH.

Examples of suitable water-insoluble polymers include but are not limited to ethylcellulose, cellulose acetate, cellulose acetate butyrate, polyvinyl acetate, copolymers of ethyl acrylate and methyl methacrylate, such as EUDRAGIT® RL. EUDRAGIT®RS, EUDRAGIT® NE, etc. and mixtures thereof.

Non-limiting examples of suitable water-soluble polymers include but are not limited to polyvinylpyrrolidone (e.g., Povidone K-25), polyethylene glycol (e.g., PEG 400), hydroxypropyl methylcellulose, and hydroxypropylcellulose, etc.

Representative gastrosoluble polymers include but are not limited to polyvinylacetal diethylaminoacetate (AEA), aminoalkyl methacrylate copolymer (Eudragit EPO), alkyl methacrylate/aminoalkyl methacrylate copolymers (EUDRAGIT E 100), cellulose derivatives, such as hydroxypropylmethyl cellulose and hydroxypropyl cellulose, etc.

The present compositions can further comprise other inert excipients such as talc, magnesium stearate or similar materials that are useful additions to the coating formula as they assist in decreasing the sticky or tacky nature of the polymer. Some polymeric coatings require the addition of a plasticizer. The additional non-functional coatings and inert excipients that can be present in the composition include, but are not limited to binders, lubricants; disintegrants, pigments, colorants, flavorants, glidants, stabilization agents, pore-formers and surfactants.

As discussed herein, the enteric coating can each optionally include a plasticizer. In some cases, it may be desirable to omit a plasticizer (e.g. in order to reduce cost, reduce exposure of patients to plasticizers, etc.). One of skill in the pharmaceutical arts can select suitable grades of water-insoluble polymers and/or enteric polymers amenable to forming a coating without plasticizer. Alternatively, it may be desirable to incorporate a plasticizer into one or both of the DR layers (e.g. in order to adjust the physical properties of the respective layers, or adjust the release rate of the drug). When a plasticizer is used, a non-limiting list of suitable plasticizers includes triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides and mixtures thereof. In one embodiment, the plasticizer is triethyl citrate.

The term “coating weight” refers to the dry weight of a coating as a percentage of the weight of the substrate prior to coating. For example, a 10 mg particle coated with 1 mg coating (dry weight) has a coating weight of 10%. Coating weights can range from about 2% to 50% including 5%, 10%, 15%, 20%, 30%, 40% and all ranges therebetween.

With regard to coating weight, unless indicated otherwise, all percentages and ratios are calculated by weight based on the total composition.

Coatings can be applied, for example, via fluid bed or pan coating of drug cores or drug particles or by other known methods.

It will be apparent to a person skilled in the art that release profiles can be modified by decreasing or increasing the thickness (coating weight) of the barrier-coat or delayed release coating on the drug crystals and/or additionally applying a sustained release coating under the delayed release coating to sustain the drug release.

In some embodiments, control of the delayed release is accomplished by a controlled release polymer coating, or by incorporation of the active agent in a controlled release polymer matrix.

The oral composition of the present invention can also be referred to as a pulsatile release composition. A pulsatile release composition mimics a multiple dosing profile without repeated dosing and allows a reduction in dosing frequency as compared to that drug presented as a conventional dosage form. A pulsatile release profile thus provides two or more pulses of drug release, each with its own lag time (delayed release) or lack of lag time (i.e., immediate release). In other words, a pulsatile release profile can include an immediate release pulse (i.e., no or insignificant lag time) and one or more delayed release pulses of drug, each having a characteristic lag time, or alternatively no immediate release pulse, and two or more delayed release pulses of drug release.

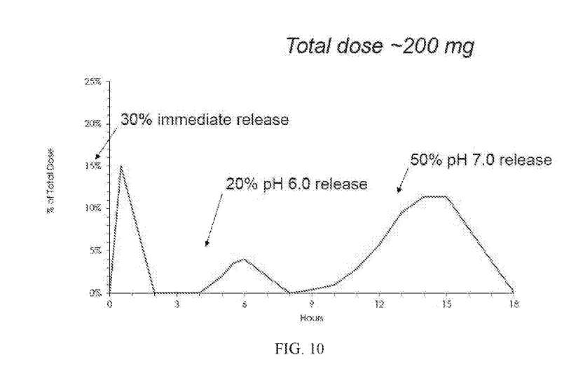

The lag time of individual pulses of the drug released from compositions having multiple pulses can be defined by the time required for an individual pulse to achieve a maximum rate of release. For example, FIG. 10 exemplifies the release characteristics of a formulation which releases the drug in 3 pulses. The maximum release of the first pulse occurs at somewhat less than about 1 hour after administration, the maximum release of the second pulse occurs about 6 hours after administration, and the maximum release of the third pulse occurs about 14-15 hours after administration. Thus, the lag times are about 1, about 6, and about 14-15 hours, respectively, for the three drug-containing components of the exemplified composition, as measured by the time from administration to maximum release, for example using suitable dissolution test conditions such as the three step dissolution test disclosed herein. In addition, the release characteristics of the compositions of the present disclosure can be defined by the lag “interval” between pulses. Alternatively, the lag time can be defined as the time (measured from administration or commencement of an appropriate dissolution test) required to achieve at least 10% release from a particular drug-containing component.

In certain embodiments, upon ingestion of the oral composition, release of the initial pulse is substantially immediate, e.g., the first salicylate drug release or “pulse” occurs within about one hour of ingestion. This initial pulse is followed by a first time interval (first lag time) during which very little or no additional drug is released from the composition, after which a second portion or drug component is then released. Similarly, a second nearly drug release-free interval (second lag time) between the second and third drug release pulses may be designed. The duration of the lag time intervals will vary depending upon the desired drug release and design of the composition. The first lag time can range from about 2 to about 7 hours (including about 2, about 3, about 4, about 5, about 6, or about 7 hours), and the second lag time can range from about 5 to about 9 hours (including about 2, about 3, about 4, about 5, about 6, about 7, about 8, or about 9 hours).

In some embodiments of the present disclosure, the initial immediate release pulse is followed by the first lag time of about 2 to about 5 hours during which very little or no additional drug is released from the composition. After the first lag time, a second (delayed release) drug component is then released. The third (delayed release) drug component is released after the second lag interval of about 2 to about 3 hours following the release of the second drug component.

In other embodiments, the initial pulse is followed by the first lag time of about 2 to about 5 and the second lag interval is about 4 to about 6 hours.

In other embodiments, the initial pulse is followed by the first lag time of about 2 to about 5 hours and the second lag interval is about 7 to about 9 hours.

In other embodiments, the initial pulse is followed by the first lag time of about 2 to about 3 hours and the second lag interval is about 2 to about 7 hours.

In other embodiments, the initial pulse is followed by the first lag time of about 2 to about 3 hours and the second lag interval is about 2 to about 4 hours.

Some embodiments relate to the oral composition providing salicylate drug release in two spaced apart pulses, where the lag time between the pulses, during which very little or no additional drug is released from the dosage form, ranges from about 2 to about 9 hours (including about 2, about 3, about 4, about 5, about 6, about 7, about 8, or about 9 hours).

Certain embodiments of the present disclosure are directed towards compositions providing controlled release of the salicylate drug in two or more pulses, for example two pulses, three pulses, or more than three pulses. In one of the embodiments, the composition provides four pulses and includes immediate release component and three different modified release components, wherein each modified release component is selected from a sustained release; delayed release, or a combination of sustained and delayed release components.

In one embodiment, the oral pharmaceutical compositions of the present invention comprise at least one IR and at least two different DR components, the IR component comprises a plurality of particles having a salicylate-releasing drug core and free of any coating which substantially delays release of salicylate; and wherein each DR component comprises a plurality of particles having the salicylate-releasing drug core coated with a DR (e.g., enteric) layer.

In some embodiments, the particles are selected from the group consisting of beads, pellets, minitabs, or mini tablets.

The drug core can be a drug particle (e.g. drug crystal).

In one of the embodiments, the immediate release particles can comprise an inert core (e.g., a sugar bead etc.) coated with a drug layer and free of any coating which substantially delays release of salicylate. In immediate release particles, the drug core can be optionally coated with a non-functional coating (coating that does not substantially delay the release of salicylate).

In one of the embodiments, the delayed release particles can comprise an inert core (e.g., a sugar bead etc.) sequentially coated with a drug layer comprising the drug and then an enteric layer.

The inert core can be any pharmaceutically acceptable inert core: in particular those with an average particle size of 10-400 μm. A non-limiting list of suitable inert cores includes sugar spheres, cellulose spheres, lactose spheres, lactose-microcrystalline (MCC) spheres, mannitol-MCC spheres, and silicon dioxide spheres.

In one of the embodiments a drug-containing core can be prepared by coating an inert particle such as a non-pareil seed, an acidic buffer crystal or an alkaline buffer crystal with a drug and a polymeric binder or by granulation and milling or by extrusion/spheronization to form an immediate release (IR) bead.

Coating the IR particle or bead with an enteric polymer will provide a delayed release (DR) particle.

The DR particles can be also prepared by granulating and milling and/or by extrusion and spheronization of an enteric polymer composition containing the IR drug particles.

In certain embodiments, the oral pharmaceutical composition of the present invention comprises one DR component wherein the pharmaceutically acceptable enteric polymer substantially dissolves at about pH 6.

In certain embodiments, the oral pharmaceutical composition of the present invention comprises one DR component wherein the pharmaceutically acceptable enteric polymer substantially dissolves at about pH 7.

In certain embodiments, the pharmaceutically acceptable enteric polymer of one DR component substantially dissolves at about pH 6, and the pharmaceutically acceptable enteric polymer of the other DR component substantially dissolves at about pH 7.

In one of the embodiments, the oral pharmaceutical composition of the present invention comprises a single IR component and two different DR components, wherein the IR particles each comprise the salicylate-releasing drug core optionally coated with a non-functional coating, one DR component comprises particles having the salicylate-releasing drug core coated with a pH-sensitive coating targeted to release salicylate at pH 6.0 and the other DR component comprises particles having the salicylate-releasing drug core coated with a pH-sensitive coating targeted to release the drug at pH 7.0.

According to certain embodiments, the amount of salicylate in the immediate release component is about 20 to about 40 wt. %, the amount of salicylate in the delayed release component coated with pharmaceutically acceptable enteric polymer that substantially dissolves at about pH 6 is about 10 to about 30 wt. %, and the amount of salicylate in the delayed release component coated with pharmaceutically acceptable enteric polymer that substantially dissolves at about pH 7 is about 40 to about 60 wt. %.

In certain embodiments, the drug or salicylate-releasing active is salsalate.

In certain embodiments, the amount of salsalate in the immediate release component is about 30 wt. %, the amount of salsalate in the delayed release component coated with pharmaceutically acceptable enteric polymer that substantially dissolves at about pH 6 is about 20 wt. %, and the amount of salsalate in the delayed release component coated with pharmaceutically acceptable enteric polymer that substantially dissolves at about pH 7 is about 50 wt. %.

The weight percent of salicylate (e.g., salsalate) is based on the total amount of salsalate in the composition.

In some embodiments, the oral composition is a multilayer particle or construct (or drug bead or granule) which has at least three layers (or components) of the salicylate-releasing drug where the outmost layer provides an immediate release and two inner layers provide different controlled (e.g., delayed) release of the drug. Each inner drug layer is coated with a controlled release coating which can be a delayed release coating (delayed release component), sustained release coating or a coating providing a combination of sustained and delayed drug releases. The outer layer releases drug substantially immediately following oral administration (immediate release component). The next inner layer has a controlled release coating surrounding the second drug layer which prevents release of the drug for about 2 to about 7 hours (including about 2, about 3, about 4, about 5, about 6, or about 7 hours). The third drug layer is coated with a different controlled release coating where it prevents release of the drug for about 2 to about 9 hours (including about 2, about 3, about 4, about 5, about 6, about 7, about 8, or about 9 hours).

In one of the embodiments of the present disclosure, the pharmaceutical composition can be a multilayered drug particle, wherein a drug particle (e.g., drug crystal, etc.) or inert core coated with a drug layer is coated with a pH-sensitive coating targeted to release the drug at pH 7.0. This construct is further coated with a second drug layer coated with a pH-sensitive coating targeted to release the drug at pH 6.0. The resulting drug particle is coated with a drug layer optionally followed by a non-functional coating. Such multilayered drug particle is functionally similar to the composition comprising a plurality of particles and therefore can provide the same PK and release profiles.

The multilayered drug particles can further comprise additional non-functional coatings and inert excipients such as binders, lubricants; disintegrants, plasticizers, pigments, colorants, flavorants, glidants, stabilization agents, pore-formers and surfactants.

Any dosage form comprising a system which allows sequential release of at least three pulses of the salicylate-releasing drug where at least one pulse is an immediate release pulse and at least two pulses have different predetermined lag times is encompassed in the present invention.

In one of the embodiments, the oral composition is formulated as a dosage form which is a capsule comprising at least one immediate release drug component and at least two different controlled or delayed release drug components.

In one of the embodiments, drug particles can be in the form of multiparticulate dosage forms (pellets, beads, granules or mini-tablets) or in other forms suitable for oral administration. As used herein, these terms are used interchangeably to refer to multiparticulate dosage forms. The drug particles of the present invention can be combined in the form of oral capsule.

In one of the embodiments, the oral composition is formulated as a dosage form which can be a capsule comprising at least two different controlled or delayed release drug components, wherein the capsule is coated with an immediate release drug component.

In one of the embodiments, the capsule can comprise drug beads generated by extrusion and spheronization or any other known process. In some embodiments, the drug particles can be granulated, compressed, or molded.

In one of the embodiments, the oral composition is formulated as a multilayered dosage form which can be a tablet comprising at least one immediate release outer drug layer and at least two different controlled or delayed release drug layers.

The multilayered drug particles or beads can also be formulated as a tablet, for example an orally disintegrating tablet (ODT). Such tablets may be more easily administered to e.g., geriatric patients as they disintegrate in the mouth and are more readily swallowed. Various types of orally disintegrating tablets are known, for example freeze-dried wafers (e.g., as used in Zydis ODT tablet compositions), tablets prepared by a loose compression process such that the resulting tablets are softer and disintegrate more rapidly, and tablets comprising the drug (e.g., a salicylate such as salsalate) and a disintegrating components such as the rapidly dispersing microgranules described om U.S. Pat. No. 8,545,881. Any suitable ODT tablet technology used in the art is suitable, provided such tablets can provide multiple drug-containing components with differing release rates as described herein.

The oral pharmaceutical composition or the oral dosage form of the present invention comprises a total dose of salicylate-releasing drug ranging from about 100 to about 1000 mg, including 200 mg, 300 mg, 400 mg, 500 mg, 600 mg, 700 mg, 800 mg, 900 mg, and all ranges therebetween.

In one of the embodiments, the total dose of salicylate-releasing drug ranges from about 200 to about 600 mg, including 250 mg, 350 mg, 450 mg, 550 mg, 650 mg, and all ranges therebetween.

In one of the embodiments, the total dose of salicylate-releasing drug ranges from about 200 to about 400 mg, including 225 mg, 250 mg, 275 mg, 300 mg, 325, 350 mg, 375 mg, and all ranges therebetween.

In one of the embodiments, the total dose of salicylate-releasing drug ranges from about 100 to about 300 mg, including 125 mg, 150 mg, 175 mg, 200 mg, 225 mg, 250 mg, 275 mg, and all ranges therebetween.

In one of the embodiments, the total dose of salicylate-releasing drug is about 200 mg.

In one of the embodiments of the present disclosure, an oral dosage form comprises an IR component having about 20-120 mg of salsalate; a first DR component with pH 6 enteric coating comprising 10-90 mg of salsalate; and a second DR component with pH 7 enteric coating comprising 40-120 mg of salsalate based on the total amount of salsalate in the dosage form of about 100-300 mg.

In one of the embodiments, a capsule contains about 60 mg of salsalate formulated as an immediate release component, about 40 mg of salsalate coated with a pH-sensitive coating targeted to release the drug at pH 6.0, and about 100 mg of salsalate coated with a pH-sensitive coating targeted to release the drug at pH 7.0. The nature of the coatings and the ratio of beads in each group can be modified by a skilled person depending on the desired clinical profile of the drug.

In some embodiments the multiparticulate composition formulated as a capsule can contain different IR and different DR components. The ratio of the components can be modified to provide the desired drug release. Upon oral administration, such a formulation provides for therapeutically effective plasma profiles over an extended period of time, thereby resulting in improved patient compliance.

Maintaining an average concentration of about 200 ng/mL of salicylate in the cerebrospinal fluid (CSF) is believed to inhibit tau acetylation and thereby slow or halt the progression of AD and other dementias. Since the concentration of salicylate in the CSF is believed to be about 10% to about 15% of the corresponding levels in plasma, an average plasma concentration of about 2 μg/mL is believed to be needed to obtain the desired CSF levels.

The average CSF concentration as used herein refers to an average value than can derive from the concentration varying between, for example, about 100 ng/mL to about 300 ng/mL or about 50 ng/mL to about 350 ng/mL, or 150 ng/mL to about 250 ng/mL etc., including about 50, about 75, about 100, about 125, about 150, about 175, about 200, about 225, about 250, about 275, or about 300 ng/mL, and all the values therebetween.

The average plasma concentration as used herein refers to an average value than can derive from the concentration varying between, for example, about 1 μg/mL to about 3 μg/mL or about 0.5 μg/mL to about 3.5 μg/mL, or 1.5 μg/mL to about 2.5 μg/mL etc., including about 0.5, about 0.75, about 1, about 1.25, about 1.5, about 1.75, about 2, about 2.25, about 2.5, about 2.75, about 3, about 3.25, or about 3.5 μg/mL, and all the values therebetween.

In some embodiments, the present disclosure is directed to oral compositions providing the average plasma concentration from about 1 μg/mL to about 3 μg/mL for a period of about 6 to about 24 hours, for example about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, inclusive of all ranges and sub ranges there between.

Some embodiments relate to maintaining the average plasma concentration over a period of 6-12 hours.

Some embodiments relate to maintaining the average plasma concentration over a period of 6-20 hours.

Certain embodiments relate to average plasma concentration being in the range of about 1 μg/mL to about 3 μg/mL for at about 24 hours.

Certain embodiments relate to average plasma concentration being in the range of about 1 μg/mL to about 3 μg/mL for at least 12 hours.

Other embodiments relate to average plasma concentration being in the range of about 1 μg/mL to about 3 μg/mL for a period of about 12-24 hours.

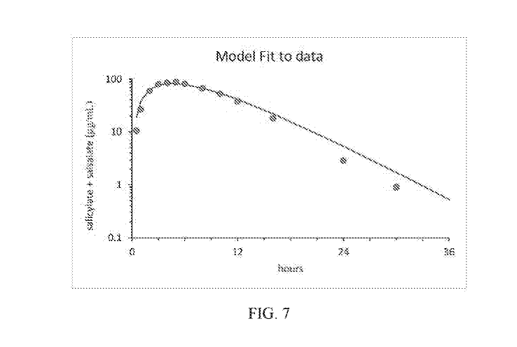

The desired average plasma concentration of about 2 μg/mL was used in a single-compartment computer model of salsalate/salicylate pharmacokinetics (PK) to determine the time course for adding drug to gastrointestinal (GI) tract to achieve the target plasma concentration. Predicted PK from this model adequately agrees with the experimental data, as shown in FIG. 7.

While it is generally assumed that constant drug levels can most readily be obtained through generation of a dosage form that provides zero order release, it was unexpectedly found that an optimal dosage form in this case would contain at least three components, at least one immediate release component and at least two delayed release components. The first and second delayed release components can be generated by applying enteric coatings, respectively, to beads, pellets, minitabs, or tablets formulated for immediate release. The immediate release component would be those beads, pellets, minitabs, or tablets without a functional (e.g., enteric) coating.

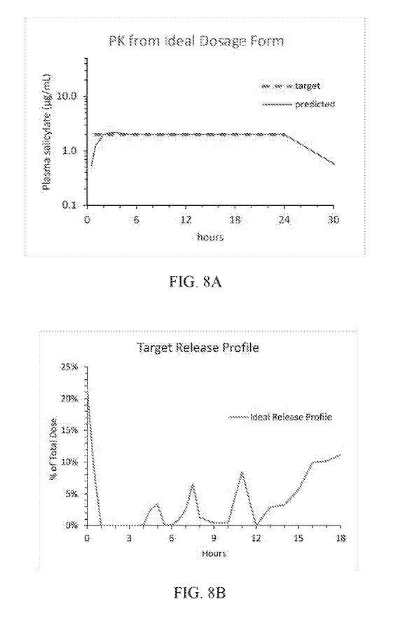

The ideal “flat” release profile for salsalate to maintain the desired PK, average plasma level of about 2 μg/mL for about 24 hours, was developed by adding a variable quantity of drug to the intestinal compartment about every thirty minutes. The residual between the target profile and the profile resulting from the model was minimized by allowing free changes in the amount of drug added at each interval. The results of this exercise are shown in FIG. 8A.

Using the computer model, an ideal release profile for salsalate was calculated to obtain the target PK (steady-state average plasma level of about 2 μg/mL) as shown in FIG. 8B, which expresses the release in terms of the percent of total drug released as a function of time.

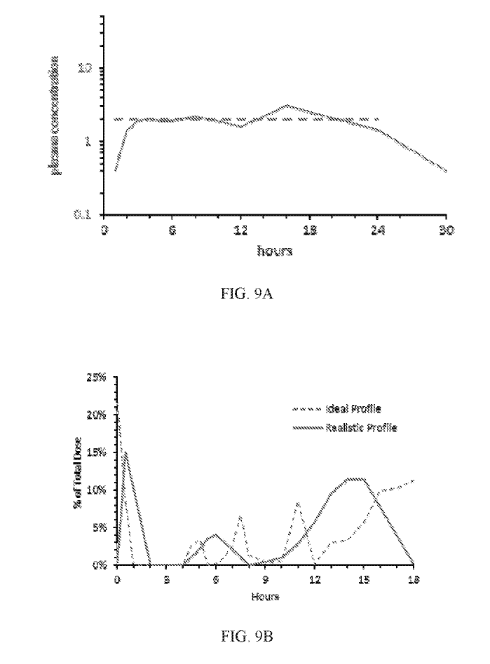

The computer modeled release profile obtained and shown in FIG. 8B may not be technically feasible using conventional controlled release technologies, so a modified “realistic” release profile that could reasonably be achieved was developed. The resulting PK and release profiles are shown in FIGS. 9A and 9B respectively.

As shown in FIG. 9B, an exemplary “realistic” release profile, that provides the desired average plasma concentration of salicylate of about 2 μg/mL for about 18-24 hours, is achieved by release of the drug in the gastrointestinal tract at about three time periods, for example, at about 1 hour, about 6 hours and at about 14-15 hours after administration. Such a release profile provides more or less constant blood plasma levels (and CSF levels) ranging from about 2 to about 24 hours post administration (FIG. 9A). The present disclosure also relates to oral compositions of salicylates which may exhibit other types of release profiles as long as they provide the desired average plasma concentration of about 2 μg/mL for at least about 12 hours. Depending on the salicylate (absorption), choice and amount of the delayed release coatings and other parameters affecting the release of the drug, the oral composition may comprise two, three, four or more delayed release or other modified release (e.g., delayed and sustained release) components to provide the desired average plasma concentration of, for example, about 2 μg/mL for at least 12 hours to maintain the desired CSF levels of salicylate to effectively inhibit tau acetylation.

After the immediate release, additional releases or pulses takes place after the predetermined lag time, which may be achieved by providing enteric coated particles, for example by coating the immediate release particles with enteric coatings having solubility at desired pH (e.g., pH 6 and/or pH 7).

In one of the embodiments, the time period for release of the drug from at least one immediate release component ranges between about 1 hour to about 3 hours.

In another embodiment, the time period for release of the drug from at least one immediate release component ranges between about 1 hour to about 6 hours.

In some embodiments, the time period for release of the drug from at least one delayed release component ranges between about 3 hour to about 6 hours (i.e, including about 3 to about 4 hours and about 3 to about 5 hours), about 3 hours to about 9 hours, or from about 3 hours to about 18 hours including all ranges therebetween.

In some embodiments, the time period for release of the drug from at least one delayed release component ranges between about 6 hour to about 9 hours, about 6 hours to about 12 hours, or from about 6 hours to about 18 hours, for example about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours, including all ranges therebetween.

In some embodiments, the time period for release of the drug from at least one delayed release component ranges between about 8 hour to about 12 hours, about 8 hours to about 18 hours, or from about 8 hours to about 24 hours, for example about 8 hours, about 9 hours, about 10 hours, about 11 hours, about 12 hours, about 13 hours, about 14 hours, about 15 hours, about 16 hours, about 17 hours, or about 18 hours, including all ranges therebetween.

In one of the embodiments, the time period for release of the drug from at least one immediate release component ranges between about 1 hour to about 3 hours, for example, about 1 hour, about 2 hours, or about 3 hours, and the time period for release of the drug from at least one delayed release component ranges between about 3 hours to about 18 hours, for example about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours, including all ranges therebetween.

In another embodiment, the time period for release of the drug from at least one immediate release component ranges between about 1 hour to about 3 hours, for example, about 1 hour, about 2 hours, or about 3 hours, and the time period for release of the drug from at least one delayed release component ranges between about 3 hours to about 9 hours, for example about 3 hours, about 4 hours, about 5 hours, about 6 hours, about 7 hours, about 8 hours, or about 9 hours, including all ranges therebetween.

In yet another embodiment, the time period for release of the drug from at least one immediate release component ranges between about 1 hour to about 3 hours, for example, about 1 hour, about 2 hours, or about 3 hours, and the time period for release of the drug from at least two different delayed release components ranges between about 3 hours to about 9 hours (for example, about 3 hours, about 4 hours, about 5 hours, about 6 hours, about 7 hours, about 8 hours, or about 9 hours) and about 9 hours to about 18 hours (for example, about 9 hours, about 10 hours, about 11 hours, about 12 hours, about 13 hours, about 14 hours, about 15 hours, about 16 hours, about 17 hours, or about 18 hours) respectively including all ranges therebetween.

In an embodiment, the composition of the present invention comprises at least one salicylate releasing delayed release component, for example comprising at least one multiparticulate salicylate releasing delayed release component. In some of these embodiments, the salicylate releasing delayed release component comprises salsalate.

In one of the embodiments, the composition of the present invention is in the form of a multiparticulate formulation of a salicylate drug which provides at least three sequential timed releases (pulses) of the salicylate drug (e.g. salsalate) to achieve the desired clinical effect (plasma level) and release profile over the time period of at least about 12 hours.

In another embodiment, the composition of the present invention provides at least three sequential timed releases (pulses) of the salicylate drug (e.g., salsalate) to achieve the steady-state average plasma concentration value of at least 2 μg/mL salicylate for at least 20 hours after administration to the subject in need thereof.

As illustrated in FIG. 10, according to the model, an exemplary composition comprising about 200 mg of salicylate drug (e.g., salsalate) releases the IR drug component comprising about 30% of the drug in less than about 3 h, followed by the pH 6 DR component comprising about 20% of the drug releasing the drug between about 4-8 hours, and then followed by the pH 7 DR component comprising about 50% of the drug and releasing the drug between about 8 and 18 hours. The first and second DR components have about 4-5 and about 8-9 hour lag time respectively from the time of administration. According to the model calculations, the desired plasma level can be achieved by releasing the drug in three consecutive pulses where the Tₘₐₓ for the IR component is about 0.5-1.5 hour, the T, for the first DR pH 6 component is about 5-7 hours and the T, for the second DR pH 7 component is about 13-15 hours. The developed model can be applied to other salicylates to optimize their release profile according to the desired clinical effect.

As disclosed hereinabove, the compositions of the present disclosure can be formulated as a composition containing two or more populations of salicylate-containing or salicylate-releasing particles, which release the salicylate at various different time points after administration (as disclosed herein) so as to provide relatively uniform plasma levels of salicylate (e.g., ranging from about 1 to about 3 g/mL) over an extended period of time (e.g. at least about 12 hours). For example, the compositions of the present disclosure can be in the form of a capsule containing three populations of salicylate-containing or salicylate-releasing particles, in which one of the populations of particles is immediate release, and the other two populations of salicylate-containing or salicylate-releasing particles are delayed release particles coated with an enteric coating, respectively dissolving at pH 5.5/6.0 and pH 7.0. Upon administration, the immediate release particles dissolve rapidly, releasing the salicylate with essentially no time lag, and the delayed release particles release the salicylate upon transiting into regions of the gastrointestinal tract having a pH at which the enteric coating is soluble. For example, when the delayed release particles having a pH 5.5 or pH 6.0 enteric coating transit into regions of the upper small intestinal tract having a pH of approximately 5.5-6.0, the pH 5.5 or pH 6.0 enteric coating dissolves, releasing the salicylate in those particles, whereas the particles coated with the pH 7.0 enteric coating remain intact until they transit further into regions of the gastrointestinal tract having a pH of approximately 7.0, whereupon the pH 7.0 enteric coating dissolves, releasing the salicylate in those particles. The respective immediate release and delayed release bead populations can each comprise a plurality of immediate release or enteric coated particles, or could contain a few mini tablets, or even a single mini tablet (e.g., a single immediate release many tablet, a single pH 5.5/6.0 enteric polymer coated mini tablet, and a single pH 7.0 enteric coated mini tablet), or combinations of a single mini tablet and a plurality of particles, provided that the appropriate amount of salicylate is contained in each population.

Alternative formulations which achieve the same plasma levels (and CSF levels) of salicylate can also be contemplated. For example, the compositions according to the present disclosure can include tablets comprising a core of immediate release salicylate drug composition (e.g., salsalate) coated with a layer of pH 7.0 enteric coating (e.g., an enteric polymer composition, optionally plasticized, which nominally dissolves at approximately pH 7) which is then further coated with a layer of salicylate drug (e.g. salsalate) and an optional binder, which is then further coated with a pH 5.5 or pH 6.0 enteric coating (e.g., an enteric polymer composition, optionally plasticized, which nominally dissolves at approximately pH 5.5 or 6.0), which is then further coated with a second layer of salicylate drug (e.g. salsalate) and optional binder. After administration, the outer salicylate drug layer is released, providing an initial, immediate release pulse of salicylate. Upon transit out of the stomach to the higher pH environment of the upper small intestine, the pH 5.5 or pH 6.0 enteric coating dissolves, releasing the salicylate drug in a second pulse of salicylate. Upon transit further down the small intestine, to higher pH environment (e.g. approximately pH 7.0), the pH 7.0 enteric coating dissolves, releasing the core layer of salicylate drug in a third pulse of salicylate. Similarly, such a tablet could be formulated as a trilayer tablet in which each layer contains salicylate drug (e.g. salsalate) in a polymeric matrix, whereby the immediate release layer comprises the salicylate drug in a readily water-soluble polymer matrix, and the delayed release layers comprise the salicylate drug in, respectively, a pH 5.5 or pH 6.0 enteric polymer, and a pH 7.0 enteric polymer. Upon administration, the readily water-soluble polymer of the immediate release layer dissolves, releasing a first pulse of salicylate drug, and the remaining delayed release layers release the salicylate drug upon reaching a suitable pH environment within the digestive tract in which the respective enteric polymers are soluble.

In still other embodiments, the compositions of the present disclosure can comprise a tablet comprising a core of an immediate release salicylate drug formulation coated with a layer of slowly eroding hydrophilic polymer, which is further coated with a layer of salicylate drug and an optional binder, which is then further coated with a second layer of slowly eroding polymer, which is then further coated with a second layer of salicylate drug and optional binder. Again, the salicylate drug is released in three separate portions or pulses as the individual hydrophilic polymer layers erode and release salicylate drug.

In another embodiment, the composition can comprise a capsule containing three tablets, one formulated for immediate release of salicylate, and the other two tablets each coated with differing amounts of slowly eroding hydrophilic polymer, or coated with a different hydrophilic polymer having differing erosion rates in an aqueous environment. The salicylate is thus released in three separate portions, an immediately release portion from the immediate release tablet, and delayed release at two different rates from the tablets coated with different hydrophilic polymer coatings. Alternatively, instead of tablets, the composition can comprise a capsule containing three different populations of salicylate drug-containing beads: a population of immediate release beads (e.g., uncoated or coated with a non-functional material) and two populations of beads each coated with a slow eroding hydrophilic polymer, differing either in the amount of hydrophilic polymer coating or the type of hydrophilic polymer to provide different release rates of salicylate from each of these populations of beads.

In still another embodiment, the composition can comprise a capsule containing three populations of salicylate drug-containing particles (or three minitablets): a population of immediate release beads (e.g., uncoated or coated with a non-functional material) and two populations of beads each coated with a swellable hydrophilic polymer, and over coated with two different insoluble, semi-permeable film compositions (e.g. ethylcellulose, and optional pore forming agent) such that upon administration the immediate release particles release the salicylate rapidly, and the remaining two populations of coated beads absorb water and swell until the different semi-permeable films rupture, releasing the salicylate from these two particle populations at different times and/or rates.

In other embodiments, the composition can comprise an osmotic pump-type tablet comprising the salicylate drug and other optional excipients in one compartment, and an osmotic agent in another compartment (or optionally the same compartment). This composition is then coated with a semipermeable coating containing a hole of well-defined diameter, such that as water diffuses through the semipermeable coating the osmotic gradient produced by the osmotic agent creates pressure that forces dissolved salicylate drug out through the whole at a defined rate. The tablet is also coated with a layer of salicylate drug and optional binder to provide an immediate release of the salicylate drug.

In yet another embodiment, the composition can comprise an osmotic pump-type tablet comprising three compartments, one of which contains an osmotic agent and the other two of which contains the salicylate drug, optionally with excipients. The compartment is coated with a semipermeable membrane containing two holes of defined diameter, one leading from each of the salsalate compartments, at least one of the holes covered with a pH-sensitive enteric polymer. The entire tablet is further coated with a layer of salicylate drug and an optional binder. Upon administration, the outer salicylate drug coating dissolves to provide immediate release, and the osmotic gradient within the tablet provides a second pulse of salicylate drug release from the uncoated hole. Upon transiting into a higher pH environment, the pH-sensitive enteric polymer coating dissolves from the remaining whole, providing a third pulse of release.

Any of the above embodiments or combinations of the above embodiments are suitable for use provided the release of salicylate results in plasma levels of salicylate ranging from about 1 to about 3 μg/mL over a period of at least about 12 hours, as described herein.

In one of the embodiments, the present invention relate to a method of inhibiting tau protein acetylation in the brain of a patient comprising orally administering the oral pharmaceutical composition to a patient in need thereof.

Some other embodiments of the present invention relate to a method of treating tauopathies and tauopathy dementias comprising administering the oral pharmaceutical composition of the present invention.

In certain embodiments, the present disclosure describes a method of treating a neurodegenerative disease or disorder associated with the pathological aggregation of tau protein in the brain comprising orally administering the oral pharmaceutical composition of the present invention to a patient in need thereof.

The present disclosure relates to the treatment of tau protein-related neurodegenerative diseases or disorders which include, but are not limited to Alzheimer's disease, Amyotrophic lateral sclerosis-Parkinsonism-dementia (ALS/PD) complex of Guam, Argyrophilic grain disease, British type amyloid angiopathy, Corticobasal degeneration, Dementia pugilistica (TBI), Down's syndrome, Frontotemporal dementia, Gerstmann-Straussler-Scheinker Syndrome, Hallenvorden-Spatz disease, Inclusion body myositis, Multisystem atrophy, Myotonic dystrophy, Niemann-Pick disease type C, Parkinson w/dementia of Guadeloupe, Pick's disease, Presenile dementia with tangles, Prion protein amyloid angiopathy, Progressive supranuclear palsy, Post-encephalitic parkinsonism. Subacute sclerosing panencephalitis, Tangle only dementia.

In one of the embodiments, a controlled release formulation of salsalate is used for the treatment of Alzheimer's disease.

In one of the embodiments, the present invention relates to a method of treating a tauopathy comprising administering to a patient in need thereof the oral pharmaceutical composition at a daily oral dose of about 100-600 mg of salicylate.

In another embodiment, the present invention relates to a method of treating a tauopathy comprising administering to a patient in need thereof the disclosed multiparticulate composition at a daily oral dose of about 200-400 mg of salicylate.

One of the embodiments, the present invention relates to a method of treating a tauopathy comprising administering to a patient in need thereof the disclosed multiparticulate composition at a daily oral dose of about 200 mg of salsalate.

The pharmaceutical composition of the present invention can be used in the manufacture of a medicament for the treatment of tauopathies, including Alzheimer's disease.

The quantity of salicylate compound in a unit dose of composition is an effective amount and is varied according to the particular treatment involved. One skilled in the art will appreciate that it is sometimes necessary to make routine variations to the dosage depending on the age and condition of the patient.

In some embodiments, salicylate drug is administered to a subject at a dose of about 100-600 mg. In some embodiments the dose is 200-400 mg. The dosage forms of the present invention are administered once daily.

In certain embodiments, the composition is administered to a subject as a single dose. In another embodiment, the composition is administered to a subject in multiple doses. Moreover, each individual dose can be administered with the same or a different dosage.

### Combination Therapy

The dosage forms of the present invention may be administered to a patient in need thereof in combination with an oral dosage form comprising another type of drug. Alternatively, one dosage form can comprise drug particles containing different drugs.

The method of the present invention can be used generally for all tauopathies.

### EXAMPLES

### Example 1 Acetylated K280 Tau in the Cerebrospinal Fluid of Alzheimer's Disease Patients

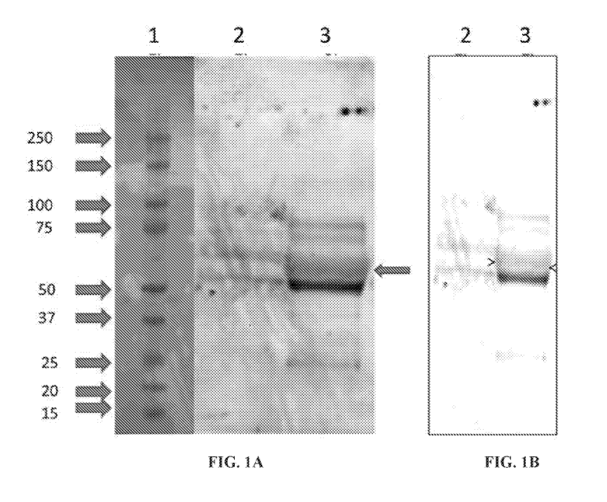

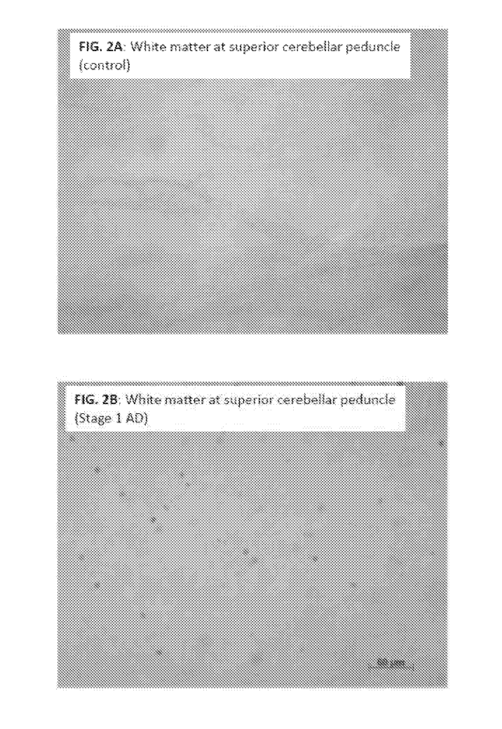

Concentrated cerebrospinal fluid samples from control and Alzheimer's disease patients were tested using Western blot analysis for the presence of acetylated K280 Tau. As shown in FIG. 1A and FIG. 2B, the AD patient sample (Lane 3) shows a faint but discernable band at the molecular weight expected for acetylated K280 Tau (about 60 kDa) when probed using the Anti-Tau Antibody (Acetyl K280), Rabbit Polyclonal available from AnaSpec (Catalog #AS-56077). On the other hand, the control experiment does not show such a band (FIG. 1A and FIG. 2B. Lane 2). Thus, putative acetylated K280 Tau has been identified in a concentrated cerebrospinal fluid sample from an Alzheimer's patient by Western blot analysis.

### Example 2 Immunohistochemical Staining of Acetylated K280 Tau in the Central Nervous System of Alzheimer's Disease Patients

Various cell types from the central nervous system of control and Alzheimer's disease patients were tested for the presence of acetylated K280 Tau using immunohistochemical staining.

The brainstem nuclei from a Braaks Stage 1 AD patient were tested for presence of acetylated K280 Tau. The following table shows semi-quantitative data (from +: mild to +++: severe staining) obtained from immunohistochemical staining using an anti-acetylated K280 Tau antibody The data indicate that acetylated K280 Tau is present in all of the AD patient brainstem nuclei tested.

| Nuclei (Relative Staining) | Nuclei (Relative Staining) |
|---|---|
| Oculomotor nerve (+) | Pontocerebellar fibres (+) |
| Crus Cerebri (++) | Trigeminal nerve (+) |
| Medial longitudinal | Internal Capsule (++) |
| fascicle (++) | external Capsule (++) |
| Medial Lemniscus (+) | White Matter of the |
| Nigrostriatal fibres (+++) | entorhinal cortex (+++) |
| Superior Cerebellar | Subcortical Cerebral |
| peduncle (+) | white matter (+++) |

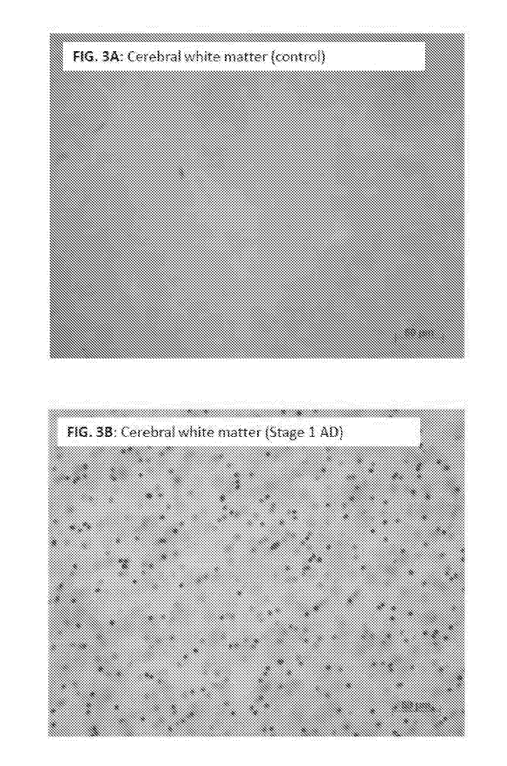

The white matter at the superior cerebellar peduncle and the cerebral white matter from Braaks Stage 1 AD and control patients were tested for presence of acetylated K280 Tau. As shown in FIGS. 2B and 3B, respectively, the superior cerebellar peduncle and cerebral white matter from the Braaks Stage 1 AD patient show staining which indicates the presence of acetylated K280 Tau. On the other hand, the control samples do not show such staining (FIG. 2A and FIG. 3A).

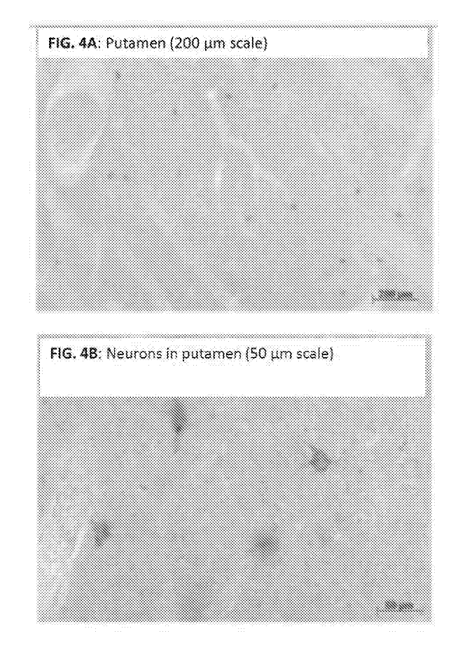

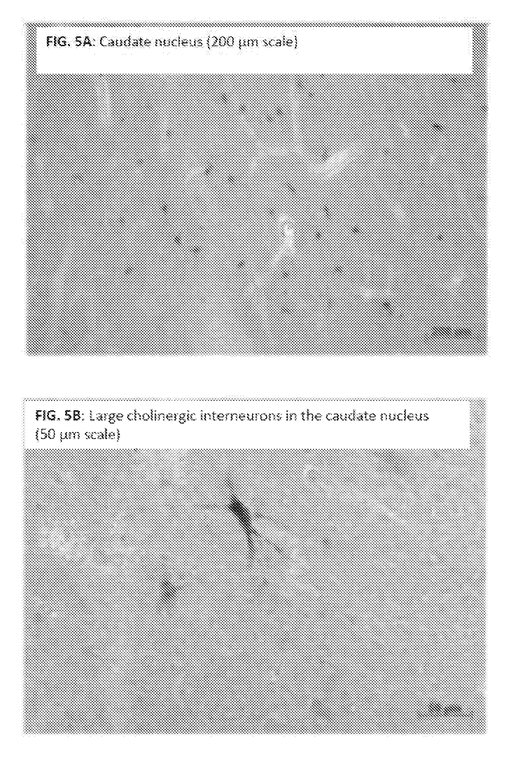

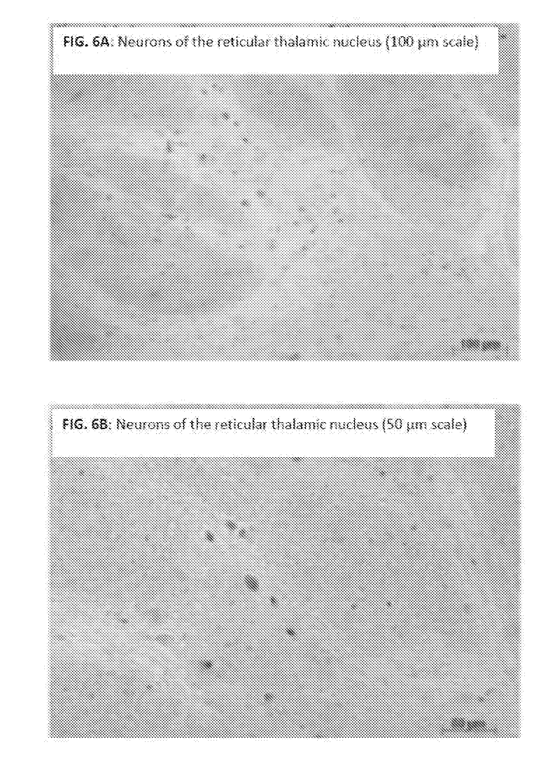

The subcortical nuclei: putamen, caudate nucleus and reticular thalamic nucleus from a Braaks Stage 1 AD were tested for presence of acetylated K280 Tau. As shown in FIG. 4A and FIG. 4B; FIG. 5A and FIG. 5B; and FIG. 6A and FIG. 6B, respectively, the putamen, caudate nucleus and reticular thalamic nucleus from the Braaks Stage 1 AD patient show distinct staining which indicates the presence of acetylated K280 Tau.

### Example 3 Salicylate IR Beads

(a) Salicylate IR beads from salicylate drug crystals: Salicylate (e.g., salsalate) drug crystals are coated with a protective seal coat of, for example, Opadry Clear for a weight gain of about 2% at a product temperature of about 50° C.

(b) Salicylate IR beads from inert cores: Salicylate (e.g., salsalate) is slowly added to purified water while stirring until dissolved. The preheated fluid bed drier (e.g., Glatt 3) is charged with inert cores, (e.g., microcrystalline cellulose spheres such as Cellets 100) and the drug solution is sprayed at a rate of, for example, about 4 mL/min with a stepwise increase to, for example, about 12 mL/min and at inlet air volume of, for example, about 8 CFM, product temperature of about 50±2° C. Following the rinse of the spray system with water, a seal coat at about 2% of Opadry Clear (about 6% solids in water) is then applied and the resulting IR beads are dried to drive off residual solvents (including moisture), and sieved (e.g., through about 40 and about 100 mesh screens) to discard oversized particles and fines.

(c) Salicylate IR beads with polymeric binder: The salicylate drug layering solution in an appropriate solvent system is prepared by first adding the polymeric binder until dissolved, followed by the salicylate drug. The solution is then applied onto inert cores, e.g., Cellets 100 (microcrystalline cellulose spheres about 100 to about 200 μm in average particle size). The resulting IR beads are then dried to drive off residual solvents (including moisture), and sieved (e.g., through about 40 and about 100 mesh screens) to discard oversized particles and fines.

### Example 4 Salicylate DR Beads

(a) Salicylate IR Beads from Example 1 are coated in a fluidized bed coating apparatus with a DR coating comprising an enteric polymer soluble at pH 6. The resulting DR coated beads are dried to drive off residual solvents.

(b) Salicylate IR Beads from Example 1 are coated in a fluidized bed coating apparatus with a DR coating comprising an enteric polymer soluble at pH 7. The resulting DR coated beads are dried to drive off residual solvents.

(c) Salicylate IR Beads from Example 1 are coated in a fluidized bed coating apparatus with a combination of DR coating comprising an enteric polymer soluble at pH 6 and optionally a sustain release coating comprising a water-insoluble polymer optionally in combination with water-soluble polymer. The resulting modified release coated beads are dried to drive off residual solvents.

(d) Salicylate IR Beads from Example 1 are coated in a fluidized bed coating apparatus with a combination of DR coating comprising an enteric polymer soluble at pH 7 and optionally a sustain release coating comprising a water-insoluble polymer optionally in combination with water-soluble polymer. The resulting modified release coated beads are dried to drive off residual solvents.

### Example 5 Exemplary Dosage Form (e.g., Capsule)

(a) Three-Component Capsule: A capsule is filled with IR beads having about 30% of salicylate and two DR components where one component comprises DR beads having about 20% of salicylate and coated with enteric coating soluble at pH 6 and the other component comprises DR beads having about 50% of salicylate and coated with enteric coating soluble at pH 7.

(b) Two-Component Coated Capsule: A capsule is filled with two DR components where one component comprises DR beads having about 20% of salicylate and coated with enteric coating soluble at pH 6 and the other component comprises DR beads having about 50% of salicylate and coated with enteric coating soluble at pH 7. This capsule if further coated with a drug coating having about 30% of salicylate and constituting the IR component of the composition.

1. An oral pharmaceutical composition comprising:
at least one salicylate-releasing immediate release component; and
at least one salicylate-releasing delayed release component;
wherein after ingestion:
the oral pharmaceutical composition maintains a plasma concentration of salicylate ranging from about 1 to about 3 μg/mL over a period of at least about 12 hours.

2. The oral pharmaceutical composition of claim 1 comprising at least two different delayed release components.

3. The oral pharmaceutical composition of claim 1 comprising a single immediate release component.

4. The oral pharmaceutical composition of claim 1 comprising a single immediate release component and two different delayed release components.

5. The oral pharmaceutical composition of claim 4, in the form of particles, wherein the two different delayed release components each comprise a plurality of particles comprising a salicylate-releasing active ingredient coated with a coating comprising a pharmaceutically acceptable enteric polymer, and the immediate release component comprises a plurality of particles comprising a salicylate-releasing active ingredient free of any coating which substantially delays release of salicylate.

6. The composition of claim 5 wherein the particles are selected from the group consisting of beads, pellets, minitabs, or mini tablets.

7. The oral pharmaceutical composition of claim 1, wherein the at least one salicylate-releasing immediate release component and the at least one salicylate-releasing delayed release component each comprise a salicylate-releasing active ingredient selected from the group consisting of salsalate, salicylic acid, and salts or esters thereof.

8. The oral pharmaceutical composition of claim 4, wherein the at least one salicylate-releasing immediate release component and the at least one salicylate-releasing delayed release component each comprise a salicylate-releasing active ingredient selected from the group consisting of salsalate, salicylic acid, and salts or esters thereof.

9. The oral pharmaceutical composition of claim 8, wherein the salicylate-releasing active ingredient is salsalate.

10. The oral pharmaceutical composition of claim 9, wherein the two different delayed release components are each coated with a coating comprising a pharmaceutically acceptable polymer which delays release of salicylate.

11. The oral pharmaceutical composition of claim 9, wherein the two different delayed release components are each coated with a coating comprising a pharmaceutically acceptable enteric polymer.

12. The oral pharmaceutical composition of claim 11, wherein the pharmaceutically acceptable enteric polymer of one delayed release component substantially dissolves at about pH 6.

13. The oral pharmaceutical composition of claim 11, wherein the pharmaceutically acceptable enteric polymer of one delayed release component substantially dissolves at about pH 7.

14. The oral pharmaceutical composition of claim 11, wherein the pharmaceutically acceptable enteric polymer of one delayed release component substantially dissolves at about pH 6, and the pharmaceutically acceptable enteric polymer of the other delayed release component substantially dissolves at about pH 7.

15. The oral pharmaceutical composition of claim 14, wherein the total dose of salsalate ranges from about 200 to about 400 mg.

16. The oral pharmaceutical composition of claim 14, wherein the amount of salsalate in the immediate release component is about 20 to about 40 wt. %, the amount of salsalate in the delayed release component coated with pharmaceutically acceptable enteric polymer that substantially dissolves at about pH 6 is about 10 to about 30 wt. %, and the amount of salsalate in the delayed release component coated with pharmaceutically acceptable enteric polymer that substantially dissolves at about pH 7 is about 40 to about 60 wt. %.

17. A method of inhibiting tau protein acetylation in the brain of a patient comprising orally administering the composition of claim 1 to a patient in need thereof.

18. A method of treating a neurodegenerative disease or disorder associated with the pathological aggregation of tau protein in the brain comprising orally administering the composition of claim 1 to a patient in need thereof.

19. The method of claim 18, wherein the neurodegenerative disease or disorder is selected from the group consisting of Alzheimer's disease, Amyotrophic lateral sclerosis-Parkinsonism-dementia (ALS/PD) complex of Guam, Argyrophilic grain disease, British type amyloid angiopathy, Corticobasal degeneration, Dementia pugilistica (TBI), Down's syndrome, Frontotemporal dementia, Gerstmann-Straussler-Scheinker, Hallenvorden-Spatz disease, Inclusion body myositis, Multisystem atrophy, Myotonic dystrophy, Niemann-Pick disease type C, Parkinson w/dementia of Guadeloupe, Pick's disease, Presenile dementia with tangles, Prion protein amyloid angiopathy, Progressive supranuclear palsy, Post-encephalitic parkinsonism, Subacute sclerosing panencephalitis, and Tangle only dementia.

20. The method of claim 19, wherein the neurodegenerative disease or disorder is Alzheimer's disease.

21. An oral pharmaceutical composition comprising at least one salicylate-releasing delayed release component.

22. The oral pharmaceutical composition of claim 21, wherein the at least one salicylate-releasing delayed release component comprises Salsalate.
